(19) United States
(12) Patent Application Publication
(10) Pub. No.: US 2025/0108053 A1
(43) Pub. Date: Apr. 3, 2025

(54) RIBOCICLIB PHARMACEUTICAL COMPOSITIONS

(71) Applicant: Novartis AG, Basel (CH)

(72) Inventors: Alpakumari CHAVDA, Hyderabad (IN); Laxman CHERKUPALLY, Hyderabad (IN); Vivek DUBEY, Hyderabad (IN); Muzammil TARIQ, Hyderabad (IN)

(21) Appl. No.: 18/730,975

(22) PCT Filed: Jan. 23, 2023
(86) PCT No.: PCT/IB2023/050559

§ 371 Date: Jul. 22, 2024

(30) Foreign Application Priority Data
Jan. 25, 2022 (IN) 202211004186

(51) Int. Cl. A61K 31/519; A61K 9/00; A61K 47/12; A61K 47/26; A61P 35/00

(52) U.S. Cl. CPC A61K 31/519; A61K 9/0095; A61K 47/12; A61K 47/26; A61P 35/00

(57) ABSTRACT

Disclosed are pharmaceutical formulations containing 7-cyclopentyl-N,N-dimethyl-2-{[5-(piperazin-1-yl)pyridin-2-yl]amino}-7H-pyrrolo[2,3-d]pyrimidine-6-carboxamide succinate, methods of using said formulations in therapy and processes for preparing the same.

### FIELD OF THE INVENTION

The present invention relates to a powder for oral solution (PFOS) comprising ribociclib and/or its pharmaceutically acceptable salts, processes for preparing the same and methods of treatment using the same. For example, the disclosure relates to a powder for oral solution (PFOS) comprising ribociclib and/or its pharmaceutically acceptable salts for use in the treatment of cancer.

### BACKGROUND OF THE INVENTION

The compound of formula (I)

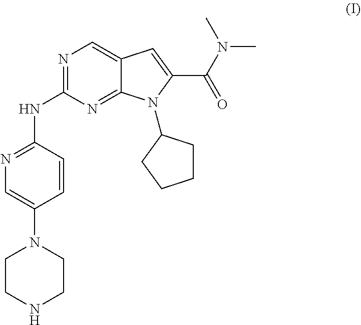 [SMILES: CN(C)C(=O)c1cc2cnc(Nc3ccc(N4CCNCC4)cn3)nc2n1C1CCCC1]

is known as ribociclib or Kisqali®. Its chemical name is 7-cyclopentyl-N,N-dimethyl-2-{[5-(piperazin-1-yl)pyridin-2-yl]amino}-7H-pyrrolo[2,3-d]pyrimidine-6-carboxamide and its synthesis is specifically described in WO 2010/020675 A1, Example 74

The succinate salt of ribociclib is described by Formula (II):

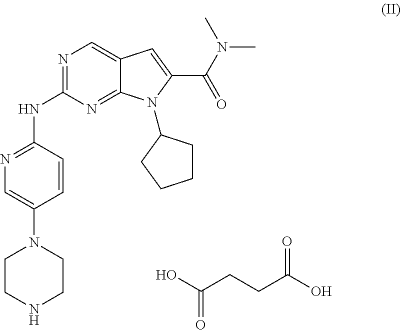 [SMILES: CN(C)C(=O)c1cc2cnc(Nc3ccc(N4CCNCC4)cn3)nc2n1C1CCCC1 ; O=C(O)CCC(=O)O]

and is described in WO2012/064805.

Ribociclib and its pharmaceutically acceptable salt(s) have valuable pharmacological properties and can be used, for example, (1) as inhibitors of cyclin dependent kinases, (in particular, cyclin dependent kinases selected from CDK1, CDK2, CDK3, CDK4, CDK5, CDK6 and CDK9); and (2) as modulators and/or inhibitors of glycogen synthase kinase-3 (GSK-3).

Solid oral pharmaceutical dosage forms are popular and useful forms of medications for dispensing pharmaceutically active compounds. A variety of such forms are known, including tablets, capsules, pellets, lozenges, and powders.

However, the formulation of an acceptable solid oral pharmaceutical dosage form on a commercial scale is not straightforward. When administered in vivo, each pharmaceutical compound acts uniquely in regards to therapeutic drug levels. Further, pharmaceutically active compounds, particularly anti-neoplastic compounds, are often associated with undesirable side effects such as; toxicity (e.g. genotoxicity, teratogenicity) and undesirable physical or psychological manifestations. In addition to balancing the drug's unique chemical properties with those of the excipients, the drug must be administered at a specific amount that is sufficient to provide the desired therapeutic drug level but less than the amount that presents an unacceptable side effect profile, or within the therapeutic window for that particular drug. Moreover, the formulation and process of manufacture must be such as to provide an integral dosage form that maintains its integrity until used. The dosage form must also possess acceptable dissolution and disintegration properties so as to provide the desired profile in use.

Pharmaceutically active compounds with low solubility and/or in solvate form can present particular challenges in preparing high quality dosage forms. These challenges include the stability and solubility of the drug substance in a pharmaceutical composition that can lead to poor pharmacodynamic properties.

Ribociclib is approved in the US, European Union, and other countries, as Kisqali®, in the form of a 200 mg film-coated tablet, for use in the treatment of advanced breast cancer in combination with an aromatase inhibitor or fulvestrant. Ribociclib is also currently being evaluated in multiple tumor types such as neuroblastoma (NB) and other solid tumors such as medulloblastoma (MB), high-grade glioma (HGG), malignant rhabdoid tumors (MRT), hepatoblastoma (HB) and rhabdomyosarcoma (RMS) in combination with temozolomide and topotecan (TOTEM).

Solid dosage forms of ribociclib, and specifically 50 mg, 100 mg, 150 mg, 200 mg and 300 mg film-coated tablets, are described in WO2016/166703. While the disclosed tablets may be acceptable for use in adults, the tablets are not preferred for administration of ribociclib to children or individuals who have difficulty swallowing tablets. In pediatric populations, it is often desired that drug be available as a powder for reconstitution to an oral suspension or solution. Such a powder requires an attempt to dry blend various excipients with the active substance in the hope of providing a powder blend with good flow properties and content uniformity.

Several additional challenges exist concerning the use of ribociclib in a pediatric formulation. For instance, ribociclib is classified as a BCS (biopharmaceutical classification system) IV compound having low solubility and low permeability. Solubility and stability challenges were encountered with aqueous solutions containing ribociclib drug substance. Ribociclib drug substance, in the form of a succinate salt, has limited solubility in basic pH as compared to acidic pH. To provide adequate physical stability and aqueous solubility, the pH of oral solutions for ribociclib succinate requires adjustment into an acidic range. In addition, oral solutions of ribociclib were found to be unstable in room temperature, requiring storage under refrigerated conditions, and having a limited shelf-life of 12 months. Further, ribociclib drug substance has been found to have a bitter taste requiring a flavored solution or suspension capable of masking taste without compromising swallowability.

Significant realization of these concerns will have an adverse effect on the in vivo administration of ribociclib.

It would be desirable to provide ribociclib in a formulation suitable for administration to a pediatric population.

### SUMMARY OF THE INVENTION

The present invention is related to a powder for oral solution (PFOS) of ribociclib, which is adapted for reconstitution with water. This invention is also related to a prepared aqueous solution, formulation, particularly to a stable oral pharmaceutical formulation, comprising ribociclib mixed with an aqueous vehicle. The present invention is related to methods of preparing these formulations. Additionally, the present invention is related to methods of treating cancer with these formulations in combination with temozolomide and topotecan. In an embodiment, the cancer is neuroblastoma (NB), medulloblastoma (MB), high-grade glioma (HGG), malignant rhabdoid tumors (MRT), hepatoblastoma (HB) and/or rhabdomyosarcoma (RMS).

### BRIEF DESCRIPTION OF THE DRAWINGS

FIG. 1 depicts process steps for preparing an oral solution containing ribociclib 30 mg/ml.

FIG. 2 depicts process steps for preparing a powder in bottle formulation containing ribociclib 30 mg/ml.

FIG. 3 depicts the study design of a phase I/II multicenter study with ribociclib in combination with topotecan and temozolomide (TOTEM) in pediatric patients with relapsed or refractory (r/r) neuroblastoma (NB) and other solid tumors.

FIG. 4 depicts the dose finding schema for Phase I—part A of the study depicted in FIG. 3.

### DETAILED DESCRIPTION

In one embodiment, the present invention is directed to oral pharmaceutical dosage forms containing ribociclib, suitably the dosage forms are powder forms, suitably the dosage forms are produced on a commercial scale. These powder forms help provide safe and effective treatment.

In one embodiment, the present invention is directed to a prepared aqueous formulation, suitably to a stable oral aqueous pharmaceutical formulation, comprising ribociclib mixed with excipients and aqueous vehicle. These prepared aqueous forms help provide safe and effective treatment.

As used herein, the term “powder for oral solution (PFOS)” means a pharmaceutical formulation containing pharmaceutical excipients and ribociclib. Prior to administration, the PFOS is reconstituted with an aqueous vehicle to form a clear or slightly colored solution. The solution is dosed based on the body weight or body surface area of the patient.

In one embodiment, the present invention is directed to powder for oral solution (PFOS) containing ribociclib in an amount of about 10 to 30%, more preferably, about 30% w/w, suitably less than 30% w/w, suitably about 25.96% w/w.

As used herein, the term “drug,” “drug substance,” or “active ingredient” and derivatives thereof, unless otherwise defined, means ribociclib or 7-cyclopentyl-N,N-dimethyl-2-{[5-(piperazin-1-yl)pyridin-2-yl]amino}-7H-pyrrolo[2,3-d]pyrimidine-6-carboxamide, preferably, in the form of a succinate salt.

By the term “commercial scale” and derivatives thereof, as used herein is meant, preparation of a batch scale greater than about 800 to 10,000 units of PFOS, suitably greater than 5000 units, suitably greater than 7500 units, or at least about 10,000 units.

It has been found that ribociclib succinate has limited solubility in basic pH as compared to acidic pH. To achieve a clear solution of ribociclib succinate upon reconstitution, sufficient quantity of acid is needed to adjust the pH of the liquid into an acidic range for ribociclib succinate to completely solubilize. For use in the present invention, suitable solubilizers are in the form of an acid.

Examples of the solubilizers, diluent/fillers, glidants, anti-adherents/lubricant, preservatives, sweeteners, and flavors are understood in the art, such components are generally described, for example, in Martindale—The Extra Pharmacopoeia Pharmaceutical Press, London (1993) and Martin (ed.), Remington's Pharmaceutical Sciences, and the Handbook of Pharmaceutical Excipients.

As used herein, “solubilizer” is a substance (liquid or solid) that helps to keep the drug uniformly dispersed and dissolved in solution. A solubilizer prevents precipitation of the dissolved drug out of solution. For use in the present invention, suitable solubilizers include, but are not limited to, citric acid, tartaric acid, glutaric acid, lactic acid, ascorbic acid, glycolic acid, mevalonic acid, malic acid, tartronic acid, maleic acid, fumaric acid, malonic acid or succinic acid. A combination of solubilizers can also be used. Citric acid was found disadvantageous due to its hygroscopic nature leading to a change in flow characteristic of the powder blend and the formation of loose lumps attributed to moisture uptake by citric acid. Surprisingly, the disadvantages of citric acid were not observed with tartaric acid, which is also hygroscopic. Suitably, the preferred solubilizer is tartaric acid.

In one embodiment of the present invention, the solubilizer(s) is (are) present in the powder for oral solution (PFOS) in an amount of about 15 to 40%, more preferably, about 25% w/w, suitably less than 25% w/w, suitably about 22.76% w/w. Preferably, the solubilizer is tartaric acid.

The term “filler” or “diluent” is used herein in its established meaning in the field of pharmaceutics, e.g. provide bulk, for example, in order to make the pharmaceutical composition a practical size for processing, or aid processing, for example, by providing improved physical properties such as flow, compressibility, and hardness. For use in the present invention, suitable diluents include, but are not limited to, microcrystalline cellulose, calcium phosphate dibasic, cellulose, lactose, sucrose, mannitol, sorbitol, starch, and calcium carbonate. Suitably, the preferred diluent is mannitol, more preferably, Mannitol SD 200.

In one embodiment of the present invention, the diluent(s) is (are) present in the powder for oral solution (PFOS) in an amount of about 10 to 60%, more preferably, about 50% w/w, suitably less than 50% w/w, suitably about 48.22% w/w. Preferably, the diluent is mannitol.

Examples of “anti-adherents” and “glidants” include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose.

In one embodiment of the present invention, the anti-adherent(s) is (are) present in the powder for oral solution (PFOS) in an amount of about 0.1 to 5%, more preferably, about 0.75% w/w, suitably less than 0.75% w/w, suitably about 0.68% w/w. Preferably, the anti-adherent is talc.

In one embodiment of the present invention, the glidant(s) is (are) present in the powder for oral solution (PFOS) in an amount of about 0.1 to 2%, more preferably, about 0.75% w/w, suitably less than 0.75% w/w, suitably about 0.68% w/w. Preferably, the glidant is colloidal silica, more preferably, anhydrous colloidal silica (e.g., Aerosil 200 PH).

As used herein, “preservative” is used to prevent the growth of bacteria and/or fungi in the liquid formulation. For instance, suitable preservatives include, but are not limited to, parabens (methyl, ethyl, propyl, and butyl), paraben sodium salt, potassium sorbate, sodium benzoate, and sorbic acid. Suitably, the amount of total preservative in a formulation according to the invention will be selected from about 0.1 to 2%, more preferably, about 0.75% w/w, suitably less than 0.75% w/w, suitably about 0.68% w/w. Preferably, the preservative is sodium benzoate.

As used herein, “sweetener” is a substance (solid or liquid) that is used to improve the palatability of the formulation. For instance, suitable sweeteners include, but are not limited to sucrose, glucose, sorbitol, saccharin sodium, aspartame, sucralose, and maltitol. Suitably, the amount of sweetener in a formulation according to the invention will be selected from about 0.1 to 2%, more preferably, about 0.5%, suitably less than 0.5% w/w, suitably about 0.34% w/w. Preferably, the sweetener is saccharin sodium.

As used herein, “flavor” is a substance (liquid or solid) that provides a distinct taste and aroma to the formulation. Flavors also help to improve the palatability of the formulation. For instance, flavors include, but are not limited to strawberry, vanilla, lemon, grape, cherry, and orange. Suitably, the flavor is orange flavor. Suitably, the amount of flavor in a formulation according to the invention will be selected from about 0.1 to 1%, more preferably, about 0.75%, suitably less than 0.75% w/w, suitably about 0.68% w/w. Preferably, the flavor is orange.

As used herein, “vehicle” is a liquid use to reconstitute a powder into an oral suspension or solution. The vehicle needs to be compatible with the formulation so that stability can be attained and maintained. For instance, suitable vehicles include, but are not limited to, purified water, sterile water for injection, and sterile water for irrigation. According to one embodiment, the vehicle is purified or sterile water.

### pH Adjustment and Preservatives

Compatible acids are required for pH adjustment and to improve solubility of ribociclib succinate without negatively affecting the stability of the formulation. A more acidic pH would help to dissolve ribociclib succinate but may be impair the solubility of other excipients such as preservatives and patient tolerance for dosage administration. The selection of preservative system, concentration of selected preservative, and pH of reconstituted liquid is determining factor for preservative efficacy.

Based on preliminary studies with ribociclib succinate in oral solutions, a clear solution was achieved within a pH range of 3.5-4.5, the preservative methyl paraben required a pH slightly above 4, and the preservative potassium sorbate requires a pH of about 3.5, and precipitation was observed in formulations with preservative sodium benzoate. While the preservative system of methyl paraben and potassium sorbate was suitable for ribociclib succinate in oral solutions, the preservative system is not suitable for powder for reconstitution to an oral solution due to poor solubility of methyl paraben and potassium sorbate. Surprisingly, it was found that sodium benzoate could be used as an effective preservative system for ribociclib succinate powder for reconstitution to an oral solution, where sufficient shaking and standing time can be used to ensure dissolution of sodium benzoate to achieve a soluble concentration.

### Flavoring and Sweeteners

The taste of ribociclib has been summarized as being bitter. The taste perception of a solution formulation was assessed by the electronic tongue (e-tongue) testing experiments.

Three different flavors (cherry, strawberry, and orange) were tested in ribociclib succinate solution formulations and its matching placebo to assess their masking efficiency. The addition of flavors was favorable to mask taste/bitterness. There was no difference detected between the tested flavors. The addition of sweeteners had a positive effect on the taste.

In one embodiment, there is provided a powder for oral solution formulation comprising:
a) about 10 to 30%, more preferably, about 30% w/w, suitably less than 30% w/w, suitably about 25.96% w/w of ribociclib succinate;
b) about 10 to 60%, more preferably, about 50% w/w, suitably less than 50% w/w, suitably about 48.22% w/w of a diluent, preferably mannitol;
c) about 15 to 40%, more preferably, about 25% w/w, suitably less than 25% w/w, suitably about 22.76% w/w of a solubilizer, preferably tartaric acid;
d) about 0.1 to 5%, more preferably, about 0.75% w/w, suitably less than 0.75% w/w, suitably about 0.68% w/w of an anti-adherent, preferably talc;
e) about 0.1 to 2%, more preferably, about 0.75% w/w, suitably less than 0.75% w/w, suitably about 0.68% w/w of a glidant, preferably colloidal silica;
f) about 0.1 to 2%; more preferably, about 0.75% w/w, suitably less than 0.75% w/w, suitably about 0.68% w/w of a preservative sodium, preferably benzoate;
g) about 0.1 to 2%; more preferably, about 0.5%, suitably less than 0.5% w/w, suitably about 0.34% w/w of a sweetener, preferably saccharin sodium; and
h) about 0.1 to 1%; more preferably, about 0.75%, suitably less than 0.75% w/w, suitably about 0.68% w/w of a flavor, preferably orange flavor.

In one embodiment, there is provided an oral solution comprising ribociclib succinate, one or more diluent(s), one or more solubilizer(s), one or more sweetener(s), one or more preservative(s), one or more anti-adherent(s), one or more glidant(s), flavor, and water.

In one embodiment, there is provided an oral solution comprising ribociclib succinate, mannitol as a diluent, tartaric acid as a solubilizer, saccharin sodium as a sweetener, sodium benzoate as a preservative, talc as an anti-adherent, colloidal silica as a glidant, orange flavor, and water.

The invented powder for oral solution (PFOS) may be administered in therapeutically effective amounts in combination with therapeutically effective amounts of other agents to treat protein kinase-associated disorders, e.g., as described in the above referenced International Publication No. WO 2010/020675.

The term “effective amount” and derivatives thereof, means that amount of a drug or active ingredient that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “co-administration” as used herein is meant either simultaneous administration or any manner of separate sequential administration of a solid or liquid oral pharmaceutical dosage form containing ribociclib, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. As used herein, “further active agent or agents” is used interchangeably with further anti-neoplastic agent or agents. Preferably, if the administration is not simultaneous, the compounds are administered in a close time proximity to each other. Furthermore, it does not matter if the compounds are administered in the same dosage form, e.g., one compound may be administered by injection and another compound may be administered orally. Suitably, the “co-administration” will consist essentially of a solid or liquid oral pharmaceutical dosage form containing ribociclib and a second pharmaceutical dosage form containing a further active agent. Suitably, the “co-administration” will consist essentially of a solid or liquid oral pharmaceutical dosage form containing ribociclib, a second pharmaceutical dosage form containing a further active agent, and a third pharmaceutical dosage form containing another further active agent.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6th edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; cell cycle signaling inhibitors; proteasome inhibitors; and inhibitors of cancer metabolism.

A method of this invention of inhibiting cyclin-dependent kinase (CDK) 4 and 6 activity in humans comprises administering to a subject in need of such inhibition a therapeutically effective amount of a powder for oral solution formulation of the present invention.

The invention also provides for the use of ribociclib in the manufacture of a powder for oral solution formulation of the present invention.

The invention also provides for the use of ribociclib in the manufacture of a powder for oral solution formulation of the present invention for use in treating cancer.

In an embodiment, the cancer is neuroblastoma (NB), medulloblastoma (MB), high-grade glioma (HGG), malignant rhabdoid tumors (MRT), hepatoblastoma (HB) and/or rhabdomyosarcoma (RMS).

In another embodiment, the cancer is relapsed or refractory neuroblastoma (NB), relapsed or refractory medulloblastoma (MB), relapsed or refractory high-grade glioma (HGG), relapsed or refractory malignant rhabdoid tumors (MRT), relapsed or refractory hepatoblastoma (HB) and/or relapsed or refractory rhabdomyosarcoma (RMS).

The invention also provides for the use of ribociclib in the manufacture of a powder for oral solution formulation of the present invention for use in inhibiting CDK.

The invention also provides for a powder for oral solution formulation for use as a CDK inhibitor, which comprises ribociclib and a pharmaceutically acceptable carrier of the present invention.

The invention also provides for a powder for oral solution formulation for use in the treatment of cancer which comprises ribociclib and a pharmaceutically acceptable carrier of the present invention.

The invention also provides for a powder for oral solution formulation for use in inhibition CDK, which comprises ribociclib and a pharmaceutically acceptable carrier of the present invention.

In one embodiment, there is provided a method for treating pediatric patients with neuroblastoma, medulloblastoma, high-grade glioma, malignant rhabdoid tumors, hepatoblastoma and/or rhabdomyosarcoma comprising administering ribociclib in a powder for oral solution formulation of the present invention in concurrent administration with topotecan and temozolomide.

In a further embodiment, there is provided a method for treating pediatric patients with relapsed or refractory neuroblastoma, relapsed or refractory medulloblastoma, relapsed or refractory high-grade glioma, relapsed or refractory malignant rhabdoid tumors, relapsed or refractory hepatoblastoma and/or relapsed or refractory rhabdomyosarcoma comprising administering ribociclib in a powder for oral solution formulation of the present invention in concurrent administration with topotecan and temozolomide. In a preferred embodiment, the pediatric patient has relapsed or refractory neuroblastoma.

In another embodiment, there is provided a method for treating pediatric patients with neuroblastoma, medulloblastoma, high-grade glioma, malignant rhabdoid tumors, hepatoblastoma and/or rhabdomyosarcoma comprising administering ribociclib in a powder for oral solution formulation of the present invention in a sequential administration with topotecan and temozolomide.

In a further embodiment, there is provided a method for treating pediatric patients with relapsed or refractory neuroblastoma, relapsed or refractory medulloblastoma, relapsed or refractory high-grade glioma, relapsed or refractory malignant rhabdoid tumors, relapsed or refractory hepatoblastoma and/or relapsed or refractory rhabdomyosarcoma comprising administering ribociclib in a powder for oral solution formulation of the present invention in sequential administration with topotecan and temozolomide. In a preferred embodiment, the pediatric patient has relapsed or refractory neuroblastoma.

In another embodiment, there is provided a method for treating pediatric patients with relapsed or refractory neuroblastoma, relapsed or refractory medulloblastoma, relapsed or refractory high-grade glioma, relapsed or refractory malignant rhabdoid tumors, relapsed or refractory hepatoblastoma and/or relapsed or refractory rhabdomyosarcoma comprising administering ribociclib succinate in a powder for oral solution formulation of the present invention at a dose of 200 mg/m2/day, 280 mg/m2/day, or 350 mg/m2/day orally on Days 1-21 out of a 28-day cycle in concurrent administration with topotecan 0.75 mg/m2/day intravenously on Days 1-5 of the 28-day cycle and temozolomide 150 mg/m2/day orally on Days 1-5 of the 28-day cycle.

In another embodiment, there is provided a method for treating pediatric patients with relapsed or refractory neuroblastoma, relapsed or refractory medulloblastoma, relapsed or refractory high-grade glioma, relapsed or refractory malignant rhabdoid tumors, relapsed or refractory hepatoblastoma and/or relapsed or refractory rhabdomyosarcoma comprising administering ribociclib succinate in a powder for oral solution formulation of the present invention at a dose of 200 mg/m2/day, 280 mg/m2/day, or 350 mg/m2/day orally on Days 6-21 out of a 28-day cycle in sequential administration with topotecan 0.75 mg/m2/day intravenously on Days 1-5 of the 28-day cycle and temozolomide 150 mg/m2/day orally on Days 1-5 of the 28-day cycle.

In another embodiment, there is provided a method for treating pediatric patients with relapsed or refractory neuroblastoma, relapsed or refractory medulloblastoma, relapsed or refractory high-grade glioma, relapsed or refractory malignant rhabdoid tumors, relapsed or refractory hepatoblastoma and/or relapsed or refractory rhabdomyosarcoma comprising administering ribociclib succinate at a dose of 200 mg/m2/day, 280 mg/m2/day, or 350 mg/m2/day orally on Days 1-21 out of a 28-day cycle in concurrent administration with topotecan 0.75 mg/m2/day intravenously on Days 1-5 of the 28-day cycle and temozolomide 150 mg/m2/day orally on Days 1-5 of the 28-day cycle.

In another embodiment, there is provided a method for treating pediatric patients with relapsed or refractory neuroblastoma, relapsed or refractory medulloblastoma, relapsed or refractory high-grade glioma, relapsed or refractory malignant rhabdoid tumors, relapsed or refractory hepatoblastoma and/or relapsed or refractory rhabdomyosarcoma comprising administering ribociclib succinate at a dose of 200 mg/m2/day, 280 mg/m2/day, or 350 mg/m2/day orally on Days 6-21 out of a 28-day cycle in sequential administration with topotecan 0.75 mg/m2/day intravenously on Days 1-5 of the 28-day cycle and temozolomide 150 mg/m2/day orally on Days 1-5 of the 28-day cycle.

Without further elaboration, it is believed that one skilled in the art can, using the preceding description, utilize the present invention to its fullest extent. Therefore, the following examples are to be construed as merely illustrative and not a limitation of the scope of the present invention.

All the excipients utilized herein are standard pharmaceutical grade excipients available from numerous manufacturers well known to those in the art.

### EXAMPLES

As used herein the symbols and conventions used in these processes, schemes and examples are consistent with those used in the contemporary scientific literature, for example, the Journal of the American Chemical Society or the Journal of Biological Chemistry. Unless otherwise indicated, all temperatures are expressed in ° C. (degrees Centigrade).

### Example 1

For the ease of swallowability, liquid formulations containing ribociclib were evaluated in the form of an oral solution, which does not require in vivo disintegration or dissolution of the drug substance that would further increase the rate of absorption.

### Formulation Preparation

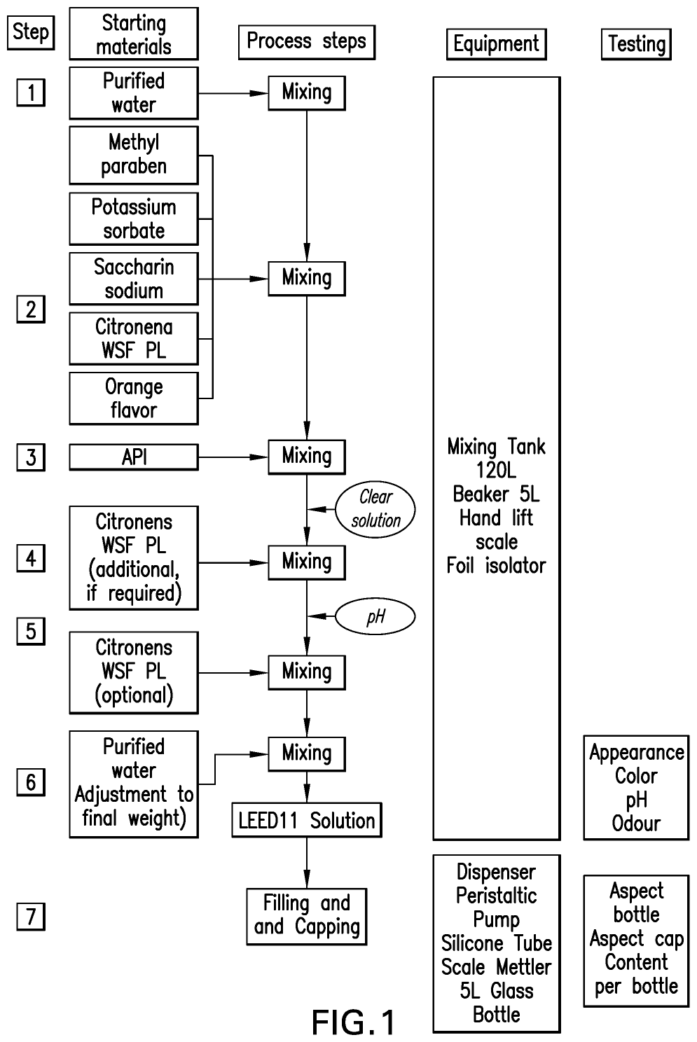

An oral solution containing ribociclib 30 mg/ml was prepared following the procedure described in the flowchart of FIG. 1. The process of Example 1 resulted in a composition having the following composition shown in Table 1.

TABLE 1

| | | Composition | Composition |
|---|---|---|---|
| | | per unit | per bottle |
| INGREDIENT | FUNCTION | (mg/ml) | (mg/bottle) |
| Ribociclib succinate* | Active | 38.16 | 3816.00 |
| | substance | | |
| Methyl paraben | Preservative | 1.60 | 16000 |
| Potassium sorbate | Preservative | 2.00 | 200.00 |
| Saccharin sodium | Sweetener | 0.50 | 50.00 |
| Orange flavor | Flavor | 1.00 | 100.00 |
| Citric Acid | Solubilizer | 33.45 | 3345.00 |
| Purified Water | Vehicle | 947.47 | 94747.00 |
| Total | | 1024.18 | 102418.00 |

*Equivalent to 30 mg of ribociclib base

The resulting formulation contains ribociclib 30 mg/mL as a clear to opalescent, orange flavored aqueous solution. The formulation was found to be unstable at room temperature, require storage at refrigerated conditions, and have a limited shelf-life of 12 months at refrigerated storage condition of 2-8° C. The color of ribociclib solution changed from orange to dark brown after 6 months storage at room temperature. In addition, there was a significant fall in the assay of active component ribociclib after 12 months storage at 25° C./60% RH.

### Example 2

### Formulation Preparation

To overcome the stability challenges of ribociclib succinate in the aqueous solution of Example 1, a powder in bottle formulation was evaluated containing a blend of drug substance with diluent and other excipients.

Table 2 depicts a qualitatively similar formulation to Example 1. In Example 2, the more easily soluble preservative of sodium salt of methyl paraben was used instead of methyl paraben. The other change was the addition of diluent mannitol, which is readily soluble in water, to add bulk to the material and support uniform filling of the blend in glass bottles.

TABLE 2

| | | | mg/ | mg/ | % |
|---|---|---|---|---|---|
| INGREDIENT | FUNCTION | mg/g | bottle | ml | w/w |
| Ribociclib | Active | 279.23 | 763.20 | 38.16 | 27.92 |
| succinate* | substance | | | | |
| Saccharin Sodium | Sweetener | 3.66 | 10.00 | 0.50 | 0.37 |
| Citric acid | Solubilizer | 182.94 | 500.00 | 25.00 | 18.29 |
| Mannitol SD | Diluent | 513.54 | 1403.60 | 70.18 | 51.35 |
| Sodium methyl | Preservative | 13.32 | 36.40 | 1.82 | 1.33 |
| paraben | | | | | |
| Strawberry flavor | Flavor | 7.32 | 20.00 | 7.32 | 0.73 |
| Total | — | 1000.00 | 2733.20 | 136.66 | 100.00 |

*Equivalent to 30 mg of ribociclib base

Ribociclib succinate was dry blended with the rest of the excipients described in formula to form final powder for oral suspension; the powder was filled in 30 ml amber glass bottle. These bottles were stored at accelerated stability condition 40° C./75% RH (4 weeks). The physical characteristics of powder after 4 weeks at accelerated storage were compared with control sample at 2-8° C.

It was observed that the powder blend converted to hard lumps on exposure to elevated temperatures and moisture; whereas the control samples stored at 2-8° C. showed free flowing powder. The change in flow characteristic of powder and formation of loose lumps are attributed to moisture uptake by citric acid due to the hygroscopic nature of citric acid. Proper selection and sealing of container closure system are taken into account to protection of formulation from moisture during storage.

### Example 3

### Formulation Preparation

To improve the physical stability of the blend of drug substance and to address the formation of undesirable lumping attributed to citric acid in the formulation of Example 2, a two-phase system was evaluated where citric acid is separated from the blend of drug substance contained in one bottle and kept in solubilized form in another bottle containing the reconstituting vehicle and a preservative. The first phase comprises a powder-in-bottle of the blend of drug substance without citric acid as provided in in Table 3A and the second phase comprises the reconstitution solvent containing citric acid and a preservative as provided in Table 3B.

TABLE 3A

| | | | mg/bottle | |
|---|---|---|---|---|
| INGREDIENT | FUNCTION | mg/ml | 100 ml | % w/w |
| Ribociclib succinate* | Active | 38.16 | 3816.00 | 32.90 |
| | substance | | | |
| Mannitol SD200 | Diluent | 74.34 | 7434.00 | 64.09 |
| Saccharin Sodium | Sweetener | 0.50 | 50.00 | 0.43 |
| Talc | Anti-adherent | 1.00 | 100.00 | 0.86 |
| Aerosil 200 | Glidant | 1.00 | 100.00 | 0.86 |
| Strawberry flavor | Flavor | 1.00 | 100.00 | 0.86 |
| Total | | 116.00 | 11600.00 | 100.00 |

*Equivalent to 30 mg of ribociclib base

TABLE 3B

| | | | mg/bottle | |
|---|---|---|---|---|
| INGREDIENT | FUNCTION | mg/ml | 100 ml | % w/w |
| Citric acid anhydrous | Active | 33.45 | 3345.00 | 3.345 |
| | substance | | | |
| Sodium methyl | Preservative | 1.82 | 182.00 | 0.18 |
| paraben | | | | |
| Purified Water | Vehicle | q.s. | q.s. to 100 ml | NA |

A four-step manufacturing process was used for the first phase—the powder phase for reconstitution—to yield batch E002A with a batch size of 3.016 kg (260 bottles). The first process step involves dividing mannitol into two equal parts and sieving one part of mannitol with Aerosil through a 0.4 mm sieve. The second process step involves sieving strawberry flavor and sodium saccharin through a 0.4 mm sieve and adding it to the blend of step one. The third process step involves sieving the second part of mannitol with ribociclib succinate in the isolator through 0.4 mm sieve, and blended together with mixture of step two for 5 mins in Turbula. The fourth process step involves the addition of talc to the Turbula and blending the mixture again for 5 minutes. The resultant blend is filled manually into 180 ml bottles, with a fill weight of 11.443 g-11.658 g.

A four-step manufacturing process was used for the second phase—the liquid phase—to yield batch E002B with a batch size of 26 L (260 bottles). The first process step involves adding purified water to a 35 L container. The second process step involves passing sodium methyl paraben through a 0.4 mm sieve. The third process step involves dissolving sodium methyl paraben in water of step one. The fourth process step involves dissolving citric acid in the contents of step 3 and filling 100 ml±5% into 180 ml bottles.

### Example 4

### Formulation Preparation

As no desiccant can be incorporated into the powder-in-bottle formulation, a formulation depicted in Table 4 involving alternate solubilizer tartaric acid was evaluated to address the formation of undesirable lumping attributed to citric acid in the formulation of Example 2.

TABLE 4

| | | | mg/bottle | |
|---|---|---|---|---|
| INGREDIENT | FUNCTION | mg/ml | 100 ml | % w/w |
| Ribociclib succinate* | Active | 38.16 | 3816.00 | 25.96 |
| | substance | | | |
| Mannitol SD200 | Diluent | 70.07 | 7007.00 | 47.67 |
| Tartaric acid | Solubilizer | 33.45 | 3345.00 | 22.76 |
| Saccharin Sodium | Sweetener | 0.50 | 50.00 | 0.34 |
| Sodium methyl | Preservative | 1.82 | 182.00 | 1.24 |
| paraben | | | | |
| Talc | Anti-adherent | 1.00 | 100.00 | 0.68 |
| Aerosil 200 | Glidant | 1.00 | 100.00 | 0.68 |
| Strawberry flavor | Flavor | 1.00 | 100.00 | 0.68 |

*Equivalent to 30 mg of ribociclib base

The formulation depicted in Table 4 was prepared with a twelve-step manufacturing process to yield batch E005 with a batch size of 3.016 kg (260 bottles). The first process step involves sieving a half quantity of mannitol with Aerosil and tartaric acid through a 0.4 mm sieve. The second process step involves mixing the blend of step one for 5 minutes in Turbula mixer. The third process step involves dividing the blend of step two into four parts. The fourth process step involves sieving sodium methyl paraben through a 0.4 mm sieve and blending it with part 1 of the blend from step three. The fifth process step involves adding part 2 of the blend from step three in Turbula mixer containing the blend of step four and mixing for 5 minutes. The sixth process step involves adding part 3 of blend from step three in Turbula mixer containing blend of step five and mixing for 5 minutes. The seventh process step involves adding part 4 of the blend from step three in Turbula mixer containing blend of step six and mixing for 5 minutes. The eighth process step 8 involves sieving sodium saccharin and strawberry flavor through a 0.4 mm sieve. The ninth process step involves blending the contents of step seven with contents of step eight for 5 minutes in Turbula mixer. The tenth process step involves sieving remaining half quantity of mannitol and ribociclib succinate through a 0.4 mm sieve and blending with contents of step nine for 5 minutes in Turbula mixer. The eleventh process step involves the addition of talc to the blend of step ten and blending the contents mixing for 5 minutes in Turbula mixer. The twelfth step involves filling 14.7 g±0.5% of the blend in step eleven into 180 ml glass bottles.

### Example 5

### Formulation Preparation

Table 5 depicts a qualitatively similar formulation to Example 4. Both formulations were manufactured using the same unit operations and processing parameters, except tartaric acid is replaced with citric acid. Batch E004 was produced with a batch size of 2.352 kg (160 bottles).

The process of Example 5 resulted in a formulation having the composition shown in Table 5.

TABLE 5

| | | | mg/bottle | |
|---|---|---|---|---|
| INGREDIENT | FUNCTION | mg/ml | 100 ml | % w/w |
| Ribociclib succinate* | Active | 38.16 | 3816.00 | 25.96 |
| | substance | | | |
| Mannitol SD200 | Diluent | 70.07 | 7007.00 | 47.67 |
| Citric acid | Solubilizer | 33.45 | 3345.00 | 22.76 |
| Saccharin Sodium | Sweetener | 0.50 | 50.00 | 0.34 |
| Sodium methyl | Preservative | 1.82 | 182.00 | 1.24 |
| paraben | | | | |
| Talc | Anti-adherent | 1.00 | 100.00 | 0.68 |
| Aerosil 200 | Glidant | 1.00 | 100.00 | 0.68 |
| Strawberry flavor | Flavor | 1.00 | 100.00 | 0.68 |

*Equivalent to 30 mg of ribociclib base

### Example 6

### Formulation Preparation

To overcome the stability challenges of ribociclib succinate in the aqueous solution of Example 1, a solution formulation was evaluated containing antioxidant sodium metabisulfite to stabilize the drug substance in aqueous solution as depicted in Table 6.

TABLE 6

| INGREDIENT | FUNCTION | mg/ml | g/batch |
|---|---|---|---|
| Ribociclib succinate* | Active | 38.2 | 610.6 |
| | substance | | |
| Sodium methyl paraben | Preservative | 1.8 | 29.1 |
| Sodium metabisulfite | Anti-oxidant | 2.0 | 32.00 |
| Saccharin Sodium | Sweetener | 0.5 | 8.00 |
| Citric acid | Solubilizer | 33.5 | 535.2 |
| Orange Flavor | Sweetener | 1.0 | 16.0 |
| Purified Water | Vehicle | 947.3 | 15156.5 |

*Equivalent to 30 mg of ribociclib base

The formulation depicted in Table 6 was prepared with a six-step manufacturing process—to yield batch E006 with a batch size of 1.6 L (160 bottles). The first process step involves adding 90% of the total quantity of water in a stainless steel container. The second process step involves dissolving sodium metabisulfite, saccharin sodium and sodium methyl paraben in the water of step one. The third process step 3 involves adding ribociclib succinate to the contents of step two to form a uniform dispersion. The fourth step involves adding citric acid to contents of step three and stirring to form a clear solution. The fifth step involves adding orange flavor to solution of step four and adding the remaining purified water. The sixth step involves flushing the container with nitrogen gas to remove dissolved oxygen and filling the resultant solution in glass bottles.

### Example 7

### Stability Studies

The formulations of Examples 3-6 were comparatively evaluated for chemical and physical stability. Based on initial observations, the study with the formulation of Example 6 (Batch E006) was abandoned due to the high levels of impurities found. The remaining formulations (from Examples 3-5) were evaluated at various time points under storage conditions of 25° C./60% RH and 40° C./75% RH as summarized in Tables 7B, 7C, and 7D. While the two-phase system of Example 3 performed well at Month 3, the stability study of formulations of Example 3 (Batches E002A & E002B) was discontinued because a significant decrease in the assay of methyl paraben was observed after 6 months. Comparative observations of batches from Examples 4 and 5 after Month 12 are summarized in Table 7A.

TABLE 7A
Physical observations of batches E004
(Ex. 5) and E005 (Ex. 4) after Month 12

| | E004 | E005 |
|---|---|---|
| | 25° C./ | 25° C./ |
| | 60% RH | 60% RH |
| Total no. of bottles in the stability | 67 | 67 |
| chamber | | |
| No. bottles with well flowable powder - | 63 of 67 | 67 of 67 |
| no lumps visible & no cake formation in | | |
| bottle neck | | |
| No. of bottles with lumps visible | 0 of 67 | 0 of 67 |
| No. of bottles with cake formation in | 4 of 67 | 0 of 67 |
| bottle neck | | |

TABLE 7B
Stability results of Batch E004 (Ex. 5)
Stability Conditions - 40° C./75% RH, Stella caps and inverted bottles

| Time point | Initial | Week 6 | Month 3 | Month 6 |
|---|---|---|---|---|
| Appearance of | Fine, | Fine, | Fine, | Cake formation, |
| powder in | homogeneous, | homogeneous, | homogeneous, | solid stopper |
| bottles | yellowish | yellowish | yellowish | sitting in the |
| | powder or | powder or | powder or | neck of the |
| | crystals | crystals | crystals | bottle (which |
| | without | without | without | had been stored |
| | lumps | lumps | lumps | in inverted |
| | | | | position) |
| Assay of | 98.1 | 96.7 | 98.2 | 97.2 |
| Methylparaben | | | | |
| (%) | | | | |
| Assay of API | 100.8 | 98.6 | 99.5 | 99.3 |
| (%) | | | | |
| Degradation of | | | | |
| API (%) | | | | |
| RRT 0.97 | <0.1 | <0.1 | <0.1 | 0.15 |
| Impurities | 0.13 | 0.12 | 0.14 | 0.13 |
| 565/13 | | | | |
| Total (%) | 0.13 | 0.12 | 0.14 | 0.28 |

TABLE 7C
Stability results of Batch E005 (Ex. 4)
Stability Conditions - 40° C./75% RH, Stella caps and inverted bottles

| Time point | Initial | Week 6 | Month 3 | Month 6 |
|---|---|---|---|---|
| Appearance of | Fine, | Fine, | Fine, | Free-flowing |
| powder in | homogeneous, | homogeneous, | homogeneous, | yellow powder |
| bottles | yellowish | yellowish | yellowish | without lumps |
| | powder or | powder or | powder or | |
| | crystals | crystals | crystals | |
| | without | without | without | |
| | lumps | lumps | lumps | |
| Assay of | 97.6 | 96.5 | 97.3 | 96.8 |
| Methylparaben | | | | |
| (%) | | | | |
| Assay of API | 100.6 | 97.5 | 99.0 | 97.6 |
| (%) | | | | |
| Degradation of | | | | |
| API (%) | | | | |
| RRT 0.97 | <0.1 | <0.1 | <0.1 | 0.13 |
| Impurities | 0.13 | 0.12 | 0.13 | 0.12 |
| 565/13 | | | | |
| Total (%) | 0.13 | 0.12 | 0.14 | 0.28 |

TABLE 7D
Stability results of Batch E005 (Ex. 4)
Stability Conditions - 25° C./60% RH, Stella caps and inverted bottles

| Time point | Initial | Week 6 | Month 3 | Month 6 | Month 12 |
|---|---|---|---|---|---|
| Appearance of | Fine, | Fine, | Fine, | Free- | Free- |
| powder in | homogeneous, | homogeneous, | homogeneous, | flowing | flowing |
| bottles | yellowish | yellowish | yellowish | yellow | yellow |
| | powder or | powder or | powder or | powder | powder |
| | crystals | crystals | crystals | without | without |
| | without | without | without | lumps | lumps |
| | lumps | lumps | lumps | | |
| Assay of | 97.6 | 95.5 | 96.2 | 97.8 | 97.2 |
| Methylparaben | | | | | |
| (%) | | | | | |
| Assay of API | 100.6 | 98.1 | 96.8 | 98.6 | 101.3 |
| (%) | | | | | |
| Degradation of | | | | | |
| API (%) | | | | | |
| RRT 0.97 | 0.1 | <0.1 | <0.1 | 0.14 | <0.1 |
| Impurities | 0.13 | 0.12 | 0.13 | 0.12 | 0.15 |
| 565/13 | | | | | |
| Total (%) | 0.13 | 0.12 | 0.13 | 0.26 | 0.15 |

Further stability studies with formulations of Example 5 were discontinued based on observations of cake formation in bottles of Batch E004 under accelerated stability conditions at 40° C./75% RH after Month 6 (Table 7C) and under long term stability conditions of controlled room temperature (CRT) at 25° C./60% RH after 12 months (Table 7A).

### Example 8

### Formulation Preparation

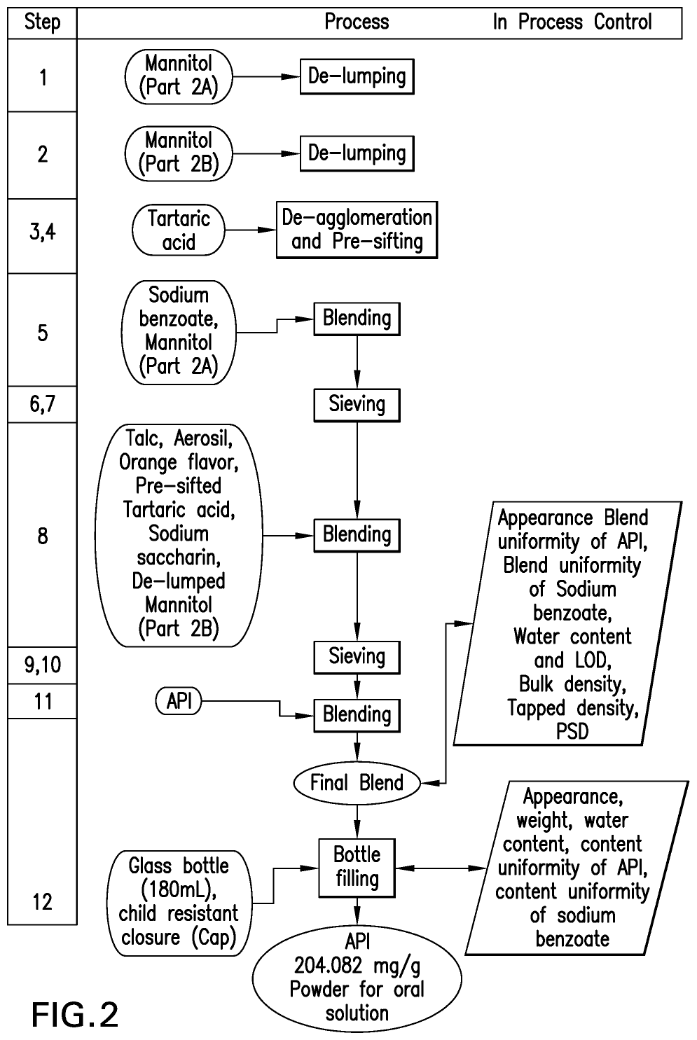

A powder in bottle formulation containing a blend of drug substance with diluent and other excipients that can be reconstituted in water to provide a ribociclib concentration of 30 mg/ml was prepared following the procedure described in the flowchart of FIG. 2. The process of Example 8 resulted in a composition having the following composition shown in Table 8.

TABLE 8

| INGREDIENT | FUNCTION | mg/g | g/bottle | % w/w |
|---|---|---|---|---|
| Ribociclib succinate¹ | Active | 259.59 | 3.816 | 25.96 |
| | substance | | | |
| Mannitol SD 200² | Diluent | 482.24 | 7.089 | 48.22 |
| Tartaric acid | Solubilizer | 227.55 | 3.345 | 22.76 |
| Saccharin sodium | Sweetener | 3.40 | 0.05 | 0.34 |
| Sodium benzoate | Preservative | 6.80 | 0.10 | 0.68 |
| Talc | Anti-adherent | 6.80 | 0.10 | 0.68 |
| Aerosil 200 PH | Glidant | 6.80 | 0.10 | 0.68 |
| Orange flavor | Flavor | 6.80 | 0.10 | 0.68 |
| Total³ | | 1000.0 | 14.70 | 100.00 |
| Purified Water | Reconstitution | — | 90 mL | — |
| | Vehicle | | | |

¹Equivalent to 3 g/bottle of ribociclib base.
²Drug substance quantity adjusted with mannitol if the content is <99.5%.
³After reconstitution with 90 ml of water per bottle for a final drug solution of 100 ml, the formulation provides a ribociclib concentration of 30 mg/ml.

The formulation depicted in Table 8 was prepared with a twelve-step manufacturing process. The first two process steps involves de-lumping two portions, parts 2A and 2B, of mannitol by sifting with a 1.0 mm screen using hand sieve or suitable equipment. Process steps three and four involve de-agglomeration of tartaric acid using 0.8 mm screen using hand sieve or suitable equipment and pre-sifting the material using 0.5 mm screen using hand sieve or suitable equipment. The fifth process step involves adding part 2A of mannitol in a suitable blender with sodium benzoate to achieve a fill volume of 25% to 70% of the container/blender and blending the contents for a total revolution of 476. The sixth process step involves screening the blend of step five through a 0.5 mm sieve. The seventh process step involves screening the materials of step six through a 0.8 mm sieve. The eighth process step involves adding the sieved material of steps six and seven to a blender of suitable size to achieve fill volume of 50% to 65%, along with talc, aerosil, orange flavor, tartaric acid (from step four) and the remaining quantity of mannitol (part 2B) and blend the contents for a total revolution of 476. The ninth process step involves sieving the blend of step eighth with 0.5 mm screen. The tenth process step involves sieving the material from step nine with a 0.8 mm screen. The eleventh process step involves adding the API drug substance to the blender of step eight, along with the sieved blend of step nine and step ten, and blending it for a total revolution of 644. The twelfth process step involves filling 180 mL amber glass bottles with the contents of step eleven using bottle filling and closing equipment.

During step twelve, in process control (IPC) evaluates the final blend against acceptance criteria for appearance, blend uniformity (BU) of ribociclib succinate, blend uniformity (BU) of preservative, water content, loss on drying (LOD), bulk density, tapped density, and particle size distribution (PSD). In process controls also evaluates bottle filling of the final blend against acceptance criteria for appearance, content uniformity (CU) of preservatives, water content, weight variation, assay of ribociclib succinate, assay of preservative, microbial enumeration test (MET), reconstitution time, and degradation products.

### Example 9

Clinical Study with Ribociclib

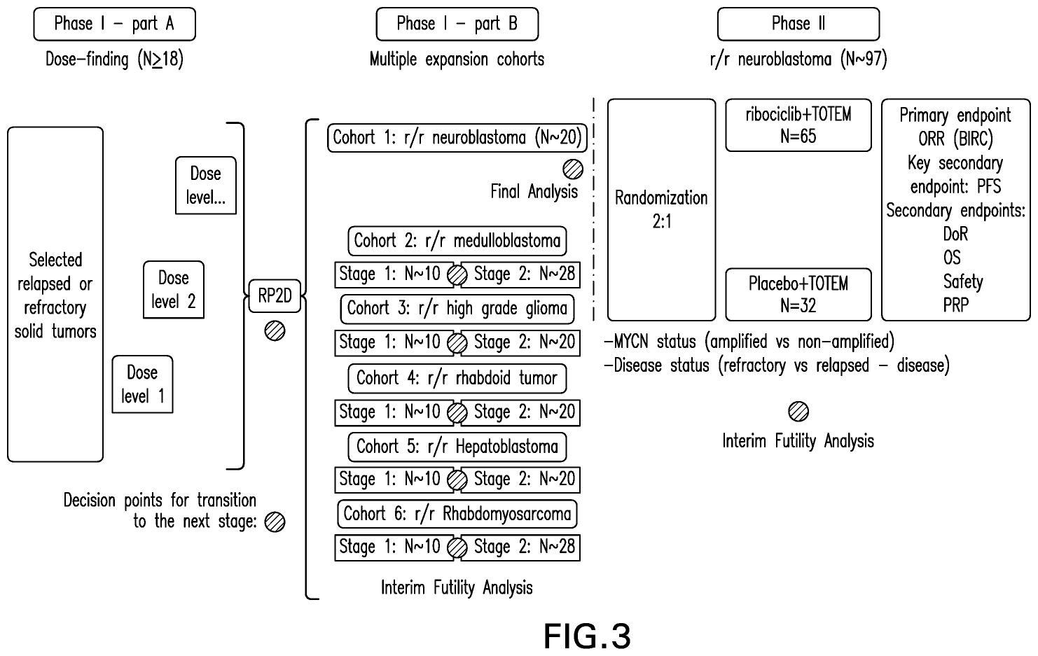

A phase I/II multicenter study to assess efficacy and safety of ribociclib in combination with topotecan and temozolomide (TOTEM) in pediatric patients with relapsed or refractory (r/r) neuroblastoma (NB) and other solid tumors, including medulloblastoma (MB), high-grade glioma (HGG), malignant rhabdoid tumors (MRT), hepatoblastoma (HB) and rhabdomyosarcoma (RMS). The study design is illustrated in FIG. 3. The study consists of a Phase I—part A (dose finding), followed by a multiple expansion cohorts' part (Phase I—part B) corresponding to r/r NB, r/r MB, r/r HCG, r/r MRT, r/r HB and r/r RMS respectively. Phase II will begin after confirmation of the anti-tumor activity in the expansion cohort in patients with r/r NB.

### Inclusion Criteria

1. Age ≥12 months and ≤21 years at the time of signing consent form
2. Histologically or cytologically confirmed solid tumors listed below that have progressed despite standard therapy or for which no effective standard therapy exists.
   a. Neuroblastoma (for Phase I and Phase II)
      i. Histologically proven neuroblastoma as per International Neuroblastoma Staging System (INSS)
      ii. Relapsed: any relapsed or progressed high-risk neuroblastoma
      iii. Refractory high-risk disease: lack of adequate response to frontline therapy that precludes the patient from proceeding to consolidation therapies (e.g. myeloblative chemotherapy)
      iv. Patients who are not the candidates for anti-GD2 therapy
      v. Measurable disease by cross sectional imaging or evaluable disease (uptake on MIBG scan with or without bone marrow histology)
      vi. Baseline scan should be obtained at least 4 weeks after the receipt of prior therapy
      vii. Patients must have an available MYCN amplification status before screening. If a local MYCN result is not available, patients must be willing to provide a tumor biopsy for central testing of MYCN amplification status.
   b. Medulloblastoma (for Phase I) Groups 3 or 4, regardless of genetic status (i.e. WNT-activated or non-WNT, SHH-activated or non-SHH)
   c. High-grade glioma (for Phase I) WHO grade III or WHO grade IV
   d. Malignant rhabdoid tumor (for Phase I): includes diagnoses of atypical teratoid/rhabdoid tumor (AT/RT), and rhabdoid tumor of the kidney (RTK), and other soft tissues as defined by 2 of the 3 following criteria; either (1)+(2) or (1)+(3):
      i. Morphology and immunophenotypic panel consistent with rhabdoid tumor
      ii. Loss of SMARCB1 confirmed by immunohistochemistry
      iii. Molecular confirmation of tumor-specific bi-allelic SMARCB1 loss/mutation is encouraged in cases where SMARCB1 immunohistochemistry is equivocal, and required if SMARCB1 immunohistochemistry is not available
   e. Hepatoblastoma (for Phase I). Note: Patients with hepatocellular carcinoma (HCC) are not eligible for the study.
   f. Rhabdomyosarcoma (for Phase I)
3. Patients with CNS disease should be on stable doses of steroids for at least 7 days prior to first dose of ribociclib with no plans for escalation
4. Evaluable or measurable disease defined by standard imaging criteria for the patient's tumor type (Response Evaluation Criteria in Solid Tumors [RECIST] v1.1 for HB, MRT (primary non-CNS Tumor) and RMS, Revised assessment in neuro-oncology [RANO] criteria for patients with MB, HGG, MRT (primary CNS Tumor) International neuroblastoma response criteria [INRC] for patients with NB)
5. Applicable to neuroblastoma patients: Patient must have an available MYCN amplification status before screening; If a local MYCN result is not available, patient must be willing to provide a tumor biopsy for central testing of MYCN amplification status.
6. Performance status: Patients who are unable to walk because of paralysis, but who are able to sit upright unassisted in a wheelchair, will be considered ambulatory for the purpose of assessing performance score
   a. ≤16 years: Lansky Play score ≥50%
   b. >16 years: Karnofsky performance status ≥50% or ECOG<3
7. Life expectancy of 12 weeks at the time of enrollment
   a. Adequate bone marrow function (bone marrow may be involved with tumor) and organ function defined as:
   b. Peripheral absolute neutrophil count (ANC) ≥1000/mm³ without growth factor support within 7 days of the test
   c. Platelet count ≥75,000/mm³ without support within 7 days of the test
   d. Hemoglobin ≥8.0 g/dL (transfusion allowed)
   e. Total bilirubin ≤1.5×ULN for age (in case of Gilbert's syndrome, ≤3.0×ULN or direct bilirubin ≤1.5×ULN for age)
   f. Adequate liver function, defined as total serum bilirubin ≤1.5×ULN AND alanine aminotransferase (ALT)/aspartate aminotransferase (AST)≤3×ULN (in case of liver metastases, AST/ALT≤5×ULN)
   g. Adequate renal function, defined as creatinine clearance or radioisotope glomerular filtration rate (GFR)≥60 mL/min/1.73 m² or serum creatinine ≤1.5×ULN based on age/gender normal
   h. Adequate cardiac function, defined as corrected QT interval (QTc)≤450 msec AND shortening fraction (SF)>29% (>35% for children <3 years) and left ventricular ejection fraction (LVEF)≥50% on echocardiogram
8. The following laboratory values within normal limits or corrected to within normal limits with supplements before first dose of study medication:
   a. Potassium
   b. Magnesium
   c. Total calcium (corrected for serum albumin)
9. Females who are sexually active must agree to use highly effective contraception during and for 6 months after treatment. Additionally, females of childbearing potential must have a negative serum pregnancy test within 7 days prior to the first dose of study medication. Pregnant or lactating females are not eligible for the study.
10. Sexually active males (including those that have had a vasectomy), who do not agree to abstinence, must be willing to use a condom during intercourse while on study treatment and for 6 months after stopping treatment.
11. Subjects and/or guardian have the ability to understand and the willingness to sign a written informed consent document.

### Dosing Regimen

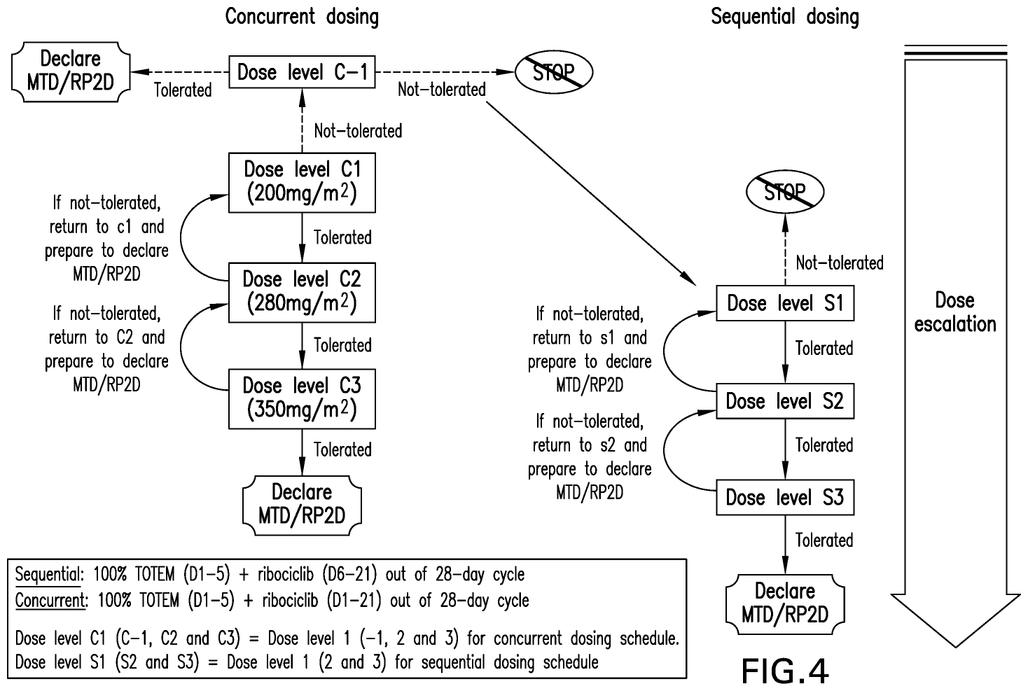

During Phase I—part A, the dose-finding phase, the ribociclib dose will be escalated while topotecan and temozolomide (TOTEM) will be administered at a fixed dose until identification of the maximum tolerated dose (MTD) and/or recommended Phase II dose (RP2D) of ribociclib in combination with TOTEM as depicted in FIG. 4.

Patients will be treated at a starting dose (dose level 1) of ribociclib 200 mg/m²/day PO (orally) on Days 1-21 out of a 28-day cycle, in concurrent administration with topotecan (0.75 mg/m²/day IV-intravenously) on Days 1-5 and temozolomide (150 mg/m²/day PO-orally on Days 1-5). The ribociclib dose will be escalated to dose level 2 (280 mg/m²/day) and dose level 3 (350 mg/m²/day) in subsequent 28-day cycles as tolerated and will continue until MTD or a suitable lower dose of ribociclib is identified for the concurrent dosing schedule. The MTD is the highest drug dosage that is not expected to cause dose-limiting toxicity (DLT) in more than 33% of the treated patients in the first cycle of ribociclib treatment in combination with TOTEM. The RP2D will be determined on the basis of comprehensive review of safety data and potentially pharmacokinetics, biomarkers and preliminary efficacy data.

If MTD/RP2D of ribociclib is not identified for the concurrent dosing schedule, despite of additional/immediate dose levels being evaluated. A sequential dosing schedule will be explored where patients will be treated with topotecan (0.75 mg/m²/day IV-intravenously) on Days 1-5 and temozolomide (150 mg/m²/day PO-orally on Days 1-5) followed by a starting a starting dose (dose level 1) of ribociclib 200 mg/m²/day PO (orally) on Days 6-21 out of a 28-day cycle. The ribociclib dose will be escalated to dose level 2 (280 mg/m²/day) and dose level 3 (350 mg/m²/day) in subsequent 28-day cycles as tolerated and will continue until MTD or a suitable lower dose of ribociclib is identified for the sequential dosing schedule. If a MTD/RP2D is identified for sequential dosing schedule, Phase I—part A dose finding will be deemed completed, and Phase I—part B will start to further evaluate this MTD/RP2D of sequential dosing schedule with more patients.

### Criteria for Starting a New Treatment Cycle

To begin a new cycle of treatment, the patient should have met the following criteria:
Peripheral absolute neutrophil count (ANC)≥1000/mm³
Platelet count ≥75,000/mm³
All non-hematologic toxicities must be ≤Grade 1 or baseline level, whichever reaches first
absence of any other DLTs as described in the DLT table

Do not start a new treatment cycle until the criteria are met:
If a new treatment cycle can begin in ≤7 days, then resume the treatment at the same dose level.
If a new treatment cycle can begin in >7-28 days, then resume the treatment at a lower dose level.
If a new treatment cycle has been delayed for more than 28 days, then discontinue the treatment.

### Criteria for Defining a Dose-Limiting Toxicity (DLT) Using CTCAE v5.0.

A dose-limiting toxicity (DLT) will be defined as an adverse event (AE) or abnormal laboratory value suspected to be related to therapy with ribociclib (i.e., assessed as unrelated to disease progression, intercurrent illness, or concomitant medications), including those AEs and abnormal laboratory values that result in failure to meet the criteria for re-treatment or to begin a new cycle of therapy within 7 days of the scheduled start date for the new cycle. The criteria for defining a DLT are listed in Table 9.

TABLE 9

| TOXICITY | Any of the following criteria |
|---|---|
| Relative dose | Patients receiving less than the 75% of the planned dose of |
| intensity | ribociclib in the first cycle for toxicity reasons |
| Hematology | Grade 4 neutropenia lasting more than 7 consecutive days or with |
| | documented infection |
| | Grade 3 or 4 thrombocytopenia requiring transfusions for more |
| | than 7 days |
| | Febrile neutropenia (Grade 3 or 4 decrease in neutrophils |
| | associated with a single temperature of >38.3° C. or a sustained |
| | temperature of ≥38° C. for more than 1 hour) |
| Renal | Serum creatinine >3 × ULN OR Serum creatinine >3 × baseline |
| Hepatobiliary | Grade 3 or 4 total bilirubin |
| | Grade 2 ALT or AST with total bilirubin >2.0 × ULN without |
| | evidence of cholestasis* (For patient with abnormal baseline AST |
| | or ALT or total bilirubin value, [ALT or AST >2 × baseline |
| | AND >3.0 × ULN] OR [ALT or AST >8.0 × ULN, whichever is |
| | lower], combined with [total bilirubin >2 × baseline AND >2.0 × |
| | ULN]) |
| | Grade 3 ALT for more than 7 consecutive days (isolated increases |
| | in AST without concomitant increases in ALT will not be |
| | considered dose-limiting, because of the non-specific nature of |
| | AST) |
| | Grade 4 ALT or AST |
| ECG QT | Average QTcF interval ≥501 ms |
| interval | OR |
| | Average QTcF interval >60 ms change from baseline |
| Gastro- | Grade 3 or 4 vomiting ≥48 hour despite optimal anti-emetic |
| intestinal | therapy (based on institutional guidelines, with consideration of |
| | the prohibited medications listed in this protocol) |
| | Grade 3 or 4 diarrhea ≥48 hour despite optimal therapy (based on |
| | institutional guidelines, with consideration of the prohibited |
| | medications listed in this protocol) |
| Non- | Grade 3 or 4 non-hematological AE except for the exclusions |
| hematological | noted below |
| events | |
| Exceptions | <5 days of Grade 3 fatigue |
| to DLT | Grade 3 fever or infection without neutropenia <5 days duration |
| criteria | Grade 3 laboratory abnormalities that are responsive to oral |
| | supplementation or deemed by the investigator to be clinically |
| | insignificant. |
| Note | CTCAE version 5.0 will be used for all grading |
| | * “Cholestasis” defined as ALP elevation (>2.0 × ULN and R |
| | value <2) in subjects without bone metastasis, or elevation of ALP |
| | liver fraction in subjects with bone metastasis. |
| | Note: The R value is calculated by dividing the ALT by the ALP, |
| | using multiples of the ULN for both values. It denotes whether the |
| | relative pattern of ALT and/or ALP elevation is due to cholestatic |
| | (R ≤2), hepatocellular (R ≥5), or mixed type R >2 and <5) liver |
| | injury. |

Treatment duration

Patients will continue the combination treatment until unacceptable toxicity, confirmed disease progression (per correspondent tumor assessment criteria), death, or discontinuation from the study treatment for any other reason (i.e. loss to follow up, subject/parents/guardian decision or withdrawal of consent) up to 12 cycles of investigational combination therapy.

After completion of 12 cycles of investigational combination therapy, in the case of ongoing clinical benefit (i.e. stable disease or better) with acceptable toxicity, patients may continue to receive ribociclib (or matching placebo for a patient enrolled in Phase II and randomized to the control arm) alone at the same dose as received in combination with TOTEM on 21-consecutive days followed by a 7-day break until disease progression, occurrence of unacceptable toxicity that precludes any further treatment.

Treatment cross-over from one arm to another arm will not be permitted in this study.

While the preferred embodiments of the invention are illustrated by the above, it is to be understood that the invention is not limited to the precise instructions herein disclosed and that the right to all modifications coming within the scope of the following claims is reserved.

What is claimed is:

1. A powder for oral solution comprising:
a) about 10% to about 30% w/w of ribociclib succinate;
b) about 10% to about 60% w/w of one or more diluent(s);
c) about 15% to about 40% w/w of one or more solubilizer(s); and
d) about 0.1% to about 2% w/w of one or more preservative(s).

2. The powder of claim 1 further comprising:
e) about 0.1% to about 5% w/w of one or more anti-adherent(s);
f) about 0.1% to about 2% w/w of one or more glidant(s);
g) about 0.1% to about 2% w/w of one or more sweetener(s); and
h) about 0.1% to about 1% w/w of a flavor.

3. The powder of claims 1 and 2, wherein the one or more diluent(s) is mannitol.

4. The powder of any one of claims 1 to 3, wherein the one or more solubilizer(s) is tartaric acid.

5. The powder of any one of claims 1 to 4, wherein the one or more preservative(s) is sodium benzoate.

6. The powder of any one of claims 2 to 5, wherein the one or more anti-adherent(s) is talc.

7. The powder of any one of claims 2 to 5, wherein the one or more anti-glidant(s) is colloidal silica.

8. The powder of any one of claims 2 to 5, wherein the one or more sweetener(s) is saccharin sodium.

9. The powder of any one of claims 2 to 5, wherein the flavor is orange.

10. An oral solution comprising:
a) about 10% to about 30% w/w of ribociclib succinate;
b) about 10% to about 60% w/w of one or more diluent(s);
c) about 15% to about 40% w/w of one or more solubilizer(s);
d) about 0.1% to about 2% w/w of one or more preservative(s); and
e) an aqueous vehicle in a quantity sufficient to bring the final volume of the oral solution to the desired volume.

11. The oral solution of claim 10, further comprising:
f) about 0.1% to about 5% w/w of one or more anti-adherent(s);
g) about 0.1% to about 2% w/w of one or more glidant(s);
h) about 0.1% to about 2% w/w of one or more sweetener(s); and
i) about 0.1% to about 1% w/w of a flavor.

12. The oral solution of claims 10 and 11, wherein the one or more diluent(s) is mannitol.

13. The oral solution of any one of claims 10 to 12, wherein the one or more solubilizer(s) is tartaric acid.

14. The oral solution of any one of claims 10 and 13, wherein the one or more preservative(s) is sodium benzoate.

15. The oral solution of any one of claims 11 to 14, wherein the one or more anti-adherent(s) is talc.

16. The oral solution of any one of claims 11 to 14, wherein the one or more anti-glidant(s) is colloidal silica.

17. The oral solution of any one of claims 11 to 14, wherein the one or more sweetener(s) is saccharin sodium

18. The oral solution any one of claims 11 to 14, wherein the flavor is orange.

19. A powder for oral solution comprising:
a) about 20% to about 30% w/w of ribociclib succinate;
b) about 40% to about 60% w/w of mannitol;
c) about 15% to about 25% w/w of tartaric acid; and
d) about 0.5% to about 1% w/w of sodium benzoate.

20. An oral solution comprising:
a) about 20% to about 30% w/w of ribociclib succinate;
b) about 40% to about 60% w/w of mannitol;
c) about 15% to about 25% w/w of tartaric acid; and
d) about 0.5% to about 1% w/w of sodium benzoate, and
e) an aqueous vehicle in a quantity sufficient to bring the final volume of the oral solution to the desired volume.

21. A formulation according to any one of claims 1 to 20 for use in the treatment of cancer in a human.

22. A formulation according to any one of claims 1 to 20 for use in inhibiting cyclin-dependent kinase (CDK) 4 and 6 in a human.
